(19) United States
(12) Patent Application Publication
(10) Pub. No.: US 2023/0233434 A1
(43) Pub. Date: Jul. 27, 2023

(54) A NO MALODOR HAIR PERM FORMULATION AND METHOD OF USE

(71) Applicant: Cali-Curl, LLC, Dallas, TX (US)

(72) Inventors: Kevin Hoye, Dallas, TX (US); David Jarvis, Reedy, WV (US)

(21) Appl. No.: 17/927,635

(22) PCT Filed: May 25, 2021
(86) PCT No.: PCT/US2021/033990

§ 371 Date: Nov. 23, 2022

Related U.S. Application Data
(60) Provisional application No. 63/029,617, filed on May 25, 2020.
(60) Provisional application No. 63/040,161, filed on Jun. 17, 2020.

(51) Int. Cl. A61K 8/46; A61K 8/06; A61K 8/22; A61K 8/34; A61K 8/92; A61Q 5/04

(52) U.S. Cl. CPC A61K 8/46; A61K 8/06; A61K 8/22; A61K 8/345; A61K 8/92; A61Q 5/04; A61K 2800/24

(57) ABSTRACT

A composition including an aqueous hair perm formulation, the aqueous hair perm formulation including an emulsion. The emulsion includes an active agent capable of reducing disulfide cysteine bond linkages in hair, an emulsifying agent, and an oil agent. A method, including producing a permanent hair wave or texture. Producing the permanent hair wave or texture includes providing a composition including an aqueous hair perm formulation, the aqueous hair perm formulation including an emulsion. The emulsion includes an active agent capable of reducing disulfide cysteine bond linkages in hair, an emulsifying agent, and an oil agent. Producing the permanent hair wave or texture also includes applying the aqueous hair perm formulation to a subject's hair.

### CROSS-REFERENCE TO RELATED APPLICATION

This application claims the benefit of U.S. Provisional Application Ser. No. 63/029,617, filed by Kevin Hoye, et al. on May 25, 2020, entitled “NO MALODOR HAIR PERM FORMULATION AND METHOD OF USE,” and U.S. Provisional Application Ser. No. 63/040,161, filed by Kevin Hoye, et al. on Jun. 17, 2020, entitled “NO MALODOR HAIR PERM FORMULATION AND METHOD OF USE”, commonly assigned with this application and incorporated herein by reference in its entirety.

### TECHNICAL FIELD

This application is directed, in general, permanent hair waving technology to create texture or waves with hair, and in particular compositions and methods to create long-lasting waves or textures in hair.

### BACKGROUND

As part of producing permanent hair waves or textures (‘perms’) some hair waving technologies use hair perm solutions including active agents to help break the disulfide bonds in hair. Ammonium thioglycolate has been used as an active agent, and, known in the industry as a “thio perm”. A negative aspect to perms, including thio perms, is that the perm solution often stinks, e.g., having a very distinct rotten egg or sulfuric smell. Additionally, thio perm solutions often have a very strong ammonia smell. Some previous efforts to mask or cover such smells in the liquid compositions have had limited success, and some such efforts can cause the compositions to lose their ability to actively break disulfide bonds due to complexation with the active agent. Additionally, fragrance agents intended to mask or cover the smell are often ineffective at sufficiently masking such odor so as not to be annoying or irritating to the client or the hair stylist. Some previous efforts to mask or cover such smells without the addition of such fragrance agents can rely on chemicals or processes that may be highly toxic or lethal to the client or stylist.

### BRIEF DESCRIPTION

Reference is now made to the following descriptions taken in conjunction with the accompanying drawings, in which:

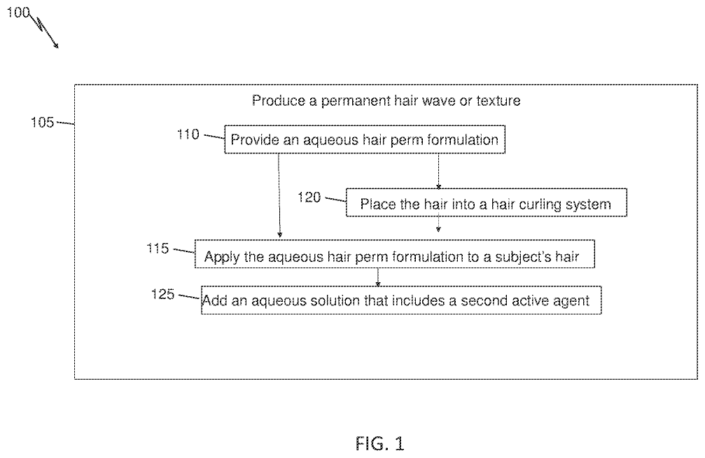

FIG. 1 presents a flow diagram of selected steps in a method embodiment of the disclosure.

### DETAILED DESCRIPTION

Embodiments of the present disclosure benefit from our discovery of a composition including an aqueous hair perm formulation that can substantially reduce the undesirable odors when using the composition to produce a permanent hair wave or texture. Our aqueous hair perm formulation includes an emulsion of an active agent capable of reducing disulfide cysteine bond linkages in hair, an emulsifying agent, and an oil agent.

Unlike previous attempts to mask the smell of malodorous chemical components of the active agent (e.g., thio or ammonia based compounds) escaping from hair perm solutions, our idea was to block the escape of malodorous chemical compounds via the emulsion. Our approach also differs from attempts to prevent the odor by complexing such malodorous chemical compounds with a complexing agent, which, by the act of complexing with the thiol functional groups of the active agent, would reduce or prevent the active agent's ability to perform its function of breaking disulfide bonds in hair.

While not limiting the scope of the disclosure by theoretical considerations, we believe that the ability of our composition to reduce the undesirable odors involves producing a steric hindrance barrier within the composition, e.g., at an air-composition interface, via the emulsion, that reduces the ability of the active agent (or chemical components of the active agent) to volatize and escape from our perm formulation into the atmosphere while at the same time not hindering the ability of the active agent to break disulfide bonds in hair. We believe that our composition, as an emulsion, has a continuous aqueous phase which includes the active agent, which can contain and react with hair to reduce the disulfide cysteine bond linkages, and, a dispersed phase of oil agent surrounded by the emulsifier that serves the barrier, e.g., via the presence of charged micelles of the oil compound in the composition. As an emulsion, our composition differs from other hair perm solutions that include an opacifer to give the appearance of being an emulsion but are actually not emulsions.

One embodiment of the disclosure is a composition including an aqueous hair perm formulation, the hair perm formulation being an emulsion of an active agent capable of reducing disulfide cysteine bond linkages in hair, an emulsifying agent, and an oil agent.

In some embodiments, the composition can further include glycerine, e.g., to give hair added moisturization and slip and thereby help promote the active agent to penetrate into the hair. In some such embodiments, the glycerine can be provided to have a concentration value in a range of about 0.5 wt % to 5 wt %, or 1 wt % to 4 wt %, or 1 wt % to 3 wt %, in the aqueous hair perm formulation and in some embodiments, a concentration value of about 2 wt %.

In some embodiments, the active agent includes or is thioglycolate (e.g., thioglycolic acid

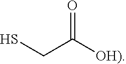

In some such embodiments, the thioglycolate can be or include an ammonia thioglycolate salt, (e.g.,

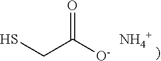

where the ammonium ion can help make hair more permeable to thioglycolate. In other embodiments the active agent can be or include other salts of thioglycolate (e.g., calcium, sodium, potassium or other thioglycolate salts). In still other embodiments, the active agent can be or include one or more of chemicals having thiol functional groups as applied to producing hair perms, as well known to those skilled in the pertinent art.

In some embodiments, the active agent can have a concentration value in a range of 1 wt % to 30 wt %, or 1 wt % to 20 wt %, or about 10 wt % to 14 wt %, or about 12 wt % in the aqueous hair perm formulation. For instance, in some embodiments, the active agent provided as ammonia thioglycolate salt (e.g., provided as a 60 wt % aqueous solution) can be in a concentration range of about 10 wt % to 14 wt %, and in some embodiments, about 12 wt %.

In some embodiments, the emulsifier agent can include or is a non-ionic emulsifier with the non-ionic emulsifier advantageously remains stable against chemical reduction in the presence of the active agent.

In some such embodiments, the non-ionic emulsifier can be one or more of polysorbate 20 (polyoxyethylene (20) sorbitan monolaurate or Tween 20) or similar chemicals well known to those skilled in the pertinent art.

In some such embodiments, the emulsifier agent can be or include one or more non-ionic emulsifiers such as polysorbate 80, polysorbate 60, ethoxylated fatty alcohols, Triton X100 (e.g., octyl phenyl ethoxylate), polyhydric alcohols, or similar chemicals well known to those skilled in the pertinent art.

In some embodiments, the emulsifier agent can be or include one or more ionic emulsifiers (e.g., cationic or anionic emulsifiers) including soaps such as potassium, sodium or other salts of fatty acids such as sodium stearate or similar chemicals well known to those skilled in the pertinent art.

In some embodiments, the emulsifier agent can include both non-ionic emulsifiers and ionic emulsifiers.

Although some ionic emulsifiers may be less stable than some non-ionic emulsifiers, the ionic emulsifier could be added to the other ingredients of the composition (e.g., one or more of active agent, oil agent and non-ionic emulsifier) to form the composition shortly before the composition is used (e.g., minutes or hours), such that the ionic emulsifiers could still facilitate forming the emulsion and block the escape of the undesirable odors during a procedure to produce a permanent hair wave or texture as disclosed herein. E.g., the composition could be configured as an “add one”-type of composition to promote shelf stability.

In some embodiments, the emulsifier agent can have a concentration value in a range of about 0.1 wt % to 15 wt %, or about 1 wt % to 10 wt % or about 3 wt % to 8 wt %, in the aqueous hair perm formulation. For instance, in some embodiments, the emulsifier agent provided as the non-ionic emulsifier provided as polysorbate 20, is in a concentration range of about 1 wt % to 8 wt % or about 3 wt % to 6 wt %.

The choice of concentration of emulsifier agent in the composition would depend on factors such as the concentration of active agent in the composition and the ability of the emulsifier to support forming and maintaining the emulsion. E.g., the concentration of emulsifier agent should be high enough to help form the emulsion but not too high so as to reduce or prevent reducing disulfide cysteine bond linkages during the time period of a hair perm procedure. For instance, in some embodiments, if the emulsifier agent (e.g., at least some ionic emulsifiers) is prone to being broken down by the active agent, and thereby less readily able to form and maintain the emulsion during a hair perm procedure, then a higher concentration of emulsifier agent may be used and/or the emulsifier agent may be added to the composition (e.g., as an “add one” component) immediately before starting the procedure.

In some embodiments, a hydrophile-lipophile balance (HLB) of the emulsifier agent in the formulation is a number in a range from 2 to 20, or from 2 to 6, or 6 to 10 or, 10 to 12, or 10 to 15, or 15 to 20, or, any combination thereof.

In some embodiments, the oil agent can be or include one or more of ester oils, such as, isopropyl palmitate, isopropyl myristate dioctyl maleate, mineral oils or similar oils know to be safe for use in various hair products, as well known to those skilled in the art.

In some embodiments, the oil agent can be or include a fatty acid, salts of the fatty acid (e.g, sodium, potassium, calcium, and/or magnesium salts) and/or an ester (e.g., methyl, ethyl, propyl, and or butyl esters) of a fatty acid. In some embodiments, the fatty acid can include or be a mono-unsaturated fatty acid and/or methyl or ethyl ester of the mono-unsaturated fatty acid. A non-limiting example mono-unsaturated fatty acid is ricinoleic acid (12-hydroxy-9-cis-octadecenoic acid) and the corresponding methyl ester and/or ethyl esters (e.g., methyl ricinoleate, ethyl ricinoleate).

In some embodiments, the mono-unsaturated fatty acid can be a double bond-terminated mono-unsaturated fatty acid have a chemical formula of:

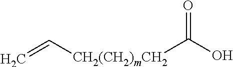

where m=0 to 11. E.g., the oil agent can be or include a terminal alkenoic acid such as: 4-Pentenoic acid (m=0), 5-Hexenoic acid (m=1), 6-Heptenoic acid (m=2), 7-Octenoic acid (m=3), 8-Nonenoic acid (m=4), 9-Decenoic acid (m=5), 10-Undecenoic acid (m=6), 11-Dodecenoic acid (m=7), 12-Tridecenoic acid (m=8) 13-Tetradecenoic acid (m=9), 14-Pentadecenoic acid (m=10), 15-hexadecenoic acid (m=11) or mixtures thereof. In some embodiments the mono-unsaturated fatty acid can be a sodium, potassium, calcium, and/or magnesium salts salt.

In some embodiments, the oil agent can be or include an ester of a mono-unsaturated fatty acid, e.g., a methyl or ethyl ester of any of the above disclosed fatty acids.

In some embodiments, the methyl ester of the mono-unsaturated fatty acid can be a methyl ester of the double-bond terminated mono-unsaturated fatty acids disclosed above and have a chemical formula of:

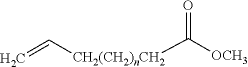

where n=0 to 11. E.g., the oil agent can be or include a terminal methyl alkenoate such as: methyl 4-Pentenoate (n=0), methyl 5-Hexenoate (n=1), methyl 6-Heptenoate (n=2), methyl 7-methyl Octenoate (n=3), methyl 8-Nonenoate (n=4), methyl 9-Decenoate (n=5), methyl 10-Undecenoate (n=6), methyl 11-Dodecenoic acid (n=7), methyl 12-Tridecenoate (n=8) methyl 13-Tetradecenoate (n=9), methyl 14-Pentadecenoate (n=10), methyl 15-Hexadecenoate (n=11) or mixtures thereof.

In some embodiments, the ethyl ester of the mono-unsaturated fatty acid can be an ethyl ester of the double bond-terminated mono-unsaturated fatty acids disclosed above and have a chemical formula of:

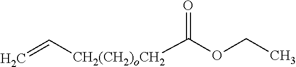

where o=0 to 11. E.g, the oil agent can be or include a terminal methyl alkenoate such as: ethyl 4-Pentenoate (o=0), ethyl 5-Hexenoate (o=1), ethyl 6-Heptenoate (o=2), ethyl 7-ethyl Octenoate (o=3), ethyl 8-Nonenoate (o=4), ethyl 9-Decenoate (o=5), ethyl 10-Undecenoate (o=6), ethyl 11-Dodecenoic acid (o=7), ethyl 12-Tridecenoate (o=8) ethyl 13-Tetradecenoate (o=9), ethyl 14-Pentadecenoate (o=10), ethyl 15-Hexadecenoate (o=11) or mixtures thereof.

Based on the present disclosure one skilled in the art would appreciate that the oil agent can be or include propyl or butyl esters of the above described double-bond terminated mono-unsaturated fatty acids.

In some embodiments where the oil agent includes double bond-terminated mono-unsaturated fatty acid and one or both of methyl or ethyl esters of such fatty acids, the fatty acids and esters can all have a same alkyl group (CH₂)_{m,n,o} chain length, e.g., m=n=o. However in other embodiments, the alkyl group chain length of the fatty acid, and methyl and ethyl esters can be different from each other, or, in some embodiments, two of the alkyl group (CH₂) chain lengths can have a same chain length (e.g., of the methyl and ethyl esters) have a same alkyl group chain length; m=o) while the third alkyl chain length (e.g., of the fatty acid) is different than the other two (e.g., m≠n, m≠o).

In some embodiments, where the oil agent can include or be (e.g., consist of, or consist essentially of with less than 10 wt %, or 5 wt % or 1 wt % of other oils present) both of methyl and ethyl esters of the double bond-terminated mono-unsaturated fatty acids disclosed above, and, a weight ratio of the methyl ester to ethyl ester can range from 1:99 to 99:1, or 5:95 to 95:5, or 10:90 to 90:10, or 80:20 to 20:80, or 30:70 to 70:30 or 40:60 to 60:40, or 50:50 or combinations thereof (e.g., 10:90 to 20:80 or 20:80 to 30:70, or 40:50 to 50:50).

In some embodiments, the oil agent can include or be only one or more of the double bond-terminated mono-unsaturated fatty acids with methyl and/or ethyl and/or propyl and/or butyl esters of the double bond-terminated mono-unsaturated fatty acids disclosed herein.

In some embodiments, the oil agent can include or be undecylenic acid (e.g., 10-undecenoic acid, m=6) or salts of undecylenic acid and/or the methyl and/or ethyl and/or propyl and/or butyl ester derivative thereof, in any proportions, and further include any of the fragrances, flavors or mixtures thereof, e.g., as disclosed in U.S. Pat. No. 6,495,097 to Streit et al., which is herein incorporated by reference in its entirety.

In some embodiments, the oil agent can have a concentration value in a range of 0.1 wt % to 15 wt %, or about 1 wt % to 10 wt % or about 3 wt % to 8 wt %, in the aqueous hair perm formulation.

In some embodiments, to promote forming an emulsion, a weight ratio of the emulsifier agent to the oil agent in the aqueous hair perm formulation can be a value in a range from about 1:1 to 4:1, or 1.5:1 to 3:1, or 1.9:1 to 2:1:1.

In some embodiments, the oil agent can further be or include a fragrance agent chemical that provides pleasant odor. In embodiments, the fragrant chemical can also be an oil. Non-limiting examples include natural and botanical fragrant oils and synthetic fragrant oils.

Some embodiments of the composition can further include other components to improve the appearance of the composition or stabilize components of the composition. For example, some embodiment can include a reducing agent stabilizer. For example, the reducing agent stabilizer can be a potassium cocoyl hydrolyzed collagen, such as May Tein C NPNF (TRI-K Industries Inc, Salem, N.H.) provided at a concentration of about 0.2 to 0.3 wt % in the formulation. For example the composition can include an opacifier to give the composition a creamy white appearance. For example, the opacifier can be or include a Styrene/Acrylamide Copolymer such as OPULYN™ 303 B opacifer (Dow Chemicals) provides at a concentration of about 0.7 to 0.8 wt % in the formulation.

Another embodiment of the disclosure is a method. FIG. 1 presents a flow diagram of selected steps in a method 100 embodiment of the disclosure. Embodiments of the method include a step 105 of producing a permanent hair wave or texture. Producing the permanent hair wave or texture (step 105) includes a step 110 of providing an aqueous hair perm formulation and a step 115 of applying the aqueous hair perm formulation to a subject's hair.

The aqueous hair perm formulation provided in step 110 can include any of the embodiments of the aqueous hair perm formulation disclosed herein and include the emulsion of the active agent, the emulsifying agent, and the oil agent.

Some embodiments of the method 100 further include, before the applying of the aqueous hair perm formulation to the hair (step 115), a step 120 of placing the hair into a hair curling system, the hair curling system including structures for wrapping portions of the hair there-around and maintaining the portions of the hair in place, and then the applying of the aqueous hair perm formulation to the hair (step 115). Non-limiting examples of the hair curling system include any of the systems disclosed in U.S. provisional patent no. 62,584,332 filed Nov. 17, 2017 to Davis et al. and U.S. patent application Ser. No. 16/186,126 filed Nov. 9, 2018 to Davis et al.

Some embodiments of the method 100 further include, after the applying of the aqueous hair perm formulation to the hair (step 115), a step 125 of adding an aqueous solution that includes a second active agent, the second active agent capable of increasing the disulfide cysteine bond linkages in the hair. In some such embodiments, the second active agent can be or include hydrogen peroxide added in concentrations familiar to those skilled in the pertinent art.

In some embodiments, to avoid or reduce damage to the hair, the applying of the aqueous hair perm formulation (step 110) can be for an application time value in a range from about 1 to 30 minutes, or from about 2 to 20 minutes.

In some embodiments, to avoid or reduce damage to the hair, the applying of the aqueous hair perm formulation (step 110) can done while maintaining a temperature of the hair and the aqueous hair perm formulation at about an ambient temperature. In some such embodiments, the ambient temperature is in a range from about 60° F. to 98° F. or 65° F. to 80° F. or 68° F. to 75° F.

We have found in experiments to produce a permanent hair wave or texture using embodiments of the disclosed composition and method can produce waves or textures that can last up to 6 months with no heat needing to be applied for styling.

We conducted tests to evaluate the subjective smell of the ammonia and sulfur odors. A nonparametric small scoring system on a scale of 0 to 10 was used.

We conducted tests to evaluate the subjective smell of the ammonia and sulfur odors. A nonparametric small scoring system on a scale of 0 to 10 was used (Appendix: Table). In the Table, Oil #2 was Green Floral Fragrance Agent (No. F-203367, Intarome, Norwood, N.J.) which we believe to include an embodiment undecylenic acid and/or its methyl and ethyl ester derivatives as disclosed in the Streit et al.; Oil #4 was Isopropyl Myristate, IPM (Protameen Chemicals Inc., Totowa, N.J.) and; Oil #5 was mineral oil. The emulsifier was polysorbate 20.

Summary of Concepts

1. A composition, comprising: an aqueous hair perm formulation, the aqueous hair perm formulation including an emulsion of: an active agent capable of reducing disulfide cysteine bond linkages in hair, an emulsifying agent, and an oil agent.

2. The composition of claim 1, further including glycerine.

3. The composition of claim 1, wherein the active agent includes thioglycolate.

4. The composition of claim 3, wherein the thioglycolate is an ammonia thioglycolate salt.

5. The composition of claim 1, wherein the active agent has a concentration value in a range of 1 wt % to 30 wt % in the aqueous hair perm formulation.

6. The composition of claim 1, wherein the emulsifier agent includes a non-ionic emulsifier.

7. The composition of claim 6, wherein the non-ionic emulsifier includes polysorbate 20.

8. The composition of claim 6, wherein the emulsifier agent has a concentration value in a range of 1 wt % to 15 wt % in the aqueous hair perm formulation.

9. The composition of claim 1, wherein a hydrophile-lipophile balance (HLB) of the emulsifier agent in the formulation is a number in a range from 2 to 20.

10. The composition of claim 1, wherein the oil agent is one or more of an oil ester, mineral oil, isopropyl myristate, isopropyl palmitate, isopropyl myristate dioctyl maleate, a fatty acid, or a salt or methyl, ethyl, propyl or butyl ester of a fatty acid.

11. The composition of claim 10, the oil agent has a concentration value in a range of 1 wt % to 15 wt % in the aqueous hair perm formulation.

12. The composition of claim 1, wherein a weight ratio of the emulsifier agent to the oil agent in the aqueous hair perm formulation is a value in a range from 1:1 to 4:1.

13. A method, comprising: producing a permanent hair wave or texture, including: providing composition including an aqueous hair perm formulation, the aqueous hair perm formulation including: an emulsion of: an active agent capable of reducing disulfide cysteine bond linkages in hair, an emulsifying agent, and an oil agent; and applying the aqueous hair perm formulation to a subject's hair.

14. The method of claim 13, further including, before the applying the aqueous hair perm formulation to the hair, placing the hair into a hair curling system, the hair curling system including structures for wrapping portions of the hair there-around and maintaining the portions of the hair in place; and then the applying of the aqueous hair perm formulation to the hair.

15. The method of claim 13 further including, after the applying of the aqueous hair perm formulation to the hair, adding an aqueous solution that includes a second active agent, the second active agent capable of increasing the disulfide cysteine bond linkages in the hair.

16. The method of claim 15, wherein the second active agent includes hydrogen peroxide.

17. The method of claim 15, wherein the applying of the aqueous hair perm formulation is for an application time value in a range from about 1 to 30 minutes.

18. The method of claim 15, wherein the applying of the aqueous hair perm formulation to a subject's hair is done while maintaining a temperature of the hair and the aqueous hair perm formulation at about an ambient temperature.

19. The method of claim 18, wherein the ambient temperature is in a range from about 60° F. to 98° F.

APPENDIX: TABLE
Hair Perm Formulation Sample Testing

| Testing | | Scent | Bottle with #1 | |
|---|---|---|---|---|
| Round 1 | Process | Description | Nubond | |
| of Samples | Time | (Unshaken) | (Shaken) | On Skin |
| Control | 4 mins | 10 | 10 | 8 |
| Oil #2 @ 1% | 4 min | 8 | 8 | 0 |
| Oil #4 @ 1% | 4 min | 8 | 8 | 5 |

SMELL SCORE (Range Of 0-10 Where Zero Is No Malodor And 10 Is Very Strong Malodor)

| | | | | | | Hair | Hair | | | | | |
|---|---|---|---|---|---|---|---|---|---|---|---|---|
| | | | | Salon | On | After | After | Wet | *Rinsed | *Rinsed | Hair | Hair |
| Testing | Skin | | | Smell | Hair | Pro- | Pro- | Salon | Foam | Foam | Re- | Re- |
| Round 1 | after | | Salon | Dissi- | During | cess | cess | Tow- | Rings | Rings | wet | Wet |
| of Samples | rinse | | Smell | pation | Process | (wet) | (Dry) | els | (Wet) | (Dry) | (Wet) | (dry) |
| Control | 4 | | 8 | 30 mins + | 10 | 6 | 4 | 10 | 5 | 3 | 3 | 2 |
| Oil #2 @ 1% | 0 | | 6 | 25 mins + | 8 | 4 | 2 | 2 | 0 | 2 | 2 | 2 |
| Oil #4 @ 1% | 0 | | 6 | 25 mins + | 8 | 5 | 3 | 7 | 2 | 3 | 2 | 2 |
| | | | | | | | | | | Bottle | | |
| Testing | | | | | | | | | | with #1 | | |
| Round 2 | Process | | | | | | | | | Nubond | | |
| of Samples | Time | | | Unshaken | | | Shaken | | | (Shaken) | | |
| Oil #2 @2% | 4 min | | | 5 | | | 3 | | | 3 | | |
| Emulsifier @2% | | | | | | | | | | | | |
| Oil #2 @2% | 4 min | | | 3 | | | 2 | | | 2 | | |
| Emulsifier @4% | | | | | | | | | | | | |
| Oil #2 @3% | 4 min | | | 3 | | | 2 | | | 2 | | |
| Emulsifier @3% | | | | | | | | | | | | |
| Oil #2 @3% | 4 min | | | 3 | | | 1 | | | 1 | | |
| Emulsifier @6% | | | | | | | | | | | | |
| | | | | | On | Hair | Hair | | | | | |
| | | | | Salon | Hair | After | After | Wet | *Rinsed | *Rinsed | Hair | Hair |
| Testing | | Skin | | Smell | During | Pro- | Pro- | Salon | Foam | Foam | Re- | Re- |
| Round 2 | On | after | Salon | Dissi- | Pro- | cess | cess | Tow- | Rings | Rings | wet | Wet |
| of Samples | Skin | rinse | Smell | pation | cess | (wet) | (Dry) | els | (Wet) | (Dry) | (Wet) | (dry) |
| Oil #2 @2% | 3 | 1 | 3 | 20 mins | 2 | 2 | 2 | 1 | 2 | 1 | 2 | 2 |
| Emulsifier @2% | | | | | | | | | | | | |
| Oil #2 @2% | 2 | 1 | 2 | 20 mins | 2 | 1 | 1 | 1 | 1 | 1 | 2 | 2 |
| Emulsifier @4% | | | | | | | | | | | | |
| Oil #2 @3% | 2 | 2 | 2 | 20 mins | 2 | 2 | 2 | 1 | 0 | 0 | 2 | 1 |
| Emulsifier @3% | | | | | | | | | | | | |
| Oil #2 @3% | 0 | 0 | 1 | 15 mins | 1 | 1 | 0 | 0 | 0 | 0 | 1 | 1 |
| Emulsifier @6% | | | | | | | | | | | | |
| | | | | | | | | | | Bottle | | |
| Testing | | | | | | | | | | with #1 | | |
| Round 3 | Process | | | | | | | | | Nubond | | |
| of Samples | Time | | | Unshaken | | | Shaken | | | (Shaken) | | |
| Oil #5 @ 3% | 4 min | | | 8 | | | 6 | | | 5 | | |
| Emulsifier | | | | | | | | | | | | |
| @ 6% | | | | | | | | | | | | |
| (Mineral Oil) | | | | | | | | | | | | |
| Oil #2 @ 3% | 4 min | | | 2 | | | 2 | | | 2 | | |
| Emulsifier | | | | | | | | | | | | |
| @ 8% | | | | | | | | | | | | |
| Oil #2 @ 4% | 5 min | | | 2 | | | 1 | | | 1 | | |
| Emulsifier | | | | | | | | | | | | |
| @ 8% | | | | | | | | | | | | |
| | | | | | On | Hair | Hair | | | | | |
| | | | | Salon | Hair | After | After | Wet | *Rinsed | *Rinsed | Hair | Hair |
| Testing | | Skin | | Smell | During | Pro- | Pro- | Salon | Foam | Foam | Re- | Re- |
| Round 3 | On | after | Salon | Dissi- | Pro- | cess | cess | Tow- | Rings | Rings | wet | Wet |
| of Samples | Skin | rinse | Smell | pation | cess | (wet) | (Dry) | els | (Wet) | (Dry) | (Wet) | (dry) |
| Oil #5 @ 3% | 5 | 4 | 4 | 20 min | 5 | 0 | 0 | 4 | 1 | | | |
| Emulsifier | | | | | | | | | | | | |
| @ 6% | | | | | | | | | | | | |
| (Mineral Oil) | | | | | | | | | | | | |
| Oil #2 @ 3% | 0 | 0 | 1 | Very fast, | 2 | 0 | 0 | 0 | 0 | 0 | | |
| Emulsifier | | | | less than | | | | | | | | |
| @ 8% | | | | 10 mins | | | | | | | | |
| Oil #2 @ 4% | 0 | 0 | 0 | NA | 1 | 0 | 0 | 0 | 0 | 0 | 0 | 0 |
| Emulsifier | | | | | | | | | | | | |
| @ 8% | | | | | | | | | | | | |

Those skilled in the art to which this application relates will appreciate that other and further additions, deletions, substitutions and modifications may be made to the described embodiments.

What is claimed:

1. A composition, comprising:
an aqueous hair perm formulation, the aqueous hair perm formulation including an emulsion of:
an active agent capable of reducing disulfide cysteine bond linkages in hair,
an emulsifying agent, and
an oil agent.

2. The composition of claim 1, further including glycerine.

3. The composition of claim 1, wherein the active agent includes thioglycolate.

4. The composition of claim 3, wherein the thioglycolate is an ammonia thioglycolate salt.

5. The composition of claim 1, wherein the active agent has a concentration value in a range of 1 wt % to 30 wt % in the aqueous hair perm formulation.

6. The composition of claim 1, wherein the emulsifier agent includes a non-ionic emulsifier.

7. The composition of claim 6, wherein the non-ionic emulsifier includes polysorbate 20.

8. The composition of claim 6, wherein the emulsifier agent has a concentration value in a range of 1 wt % to 15 wt % in the aqueous hair perm formulation.

9. The composition of claim 1, wherein a hydrophile-lipophile balance (HLB) of the emulsifier agent in the formulation is a number in a range from 2 to 20.

10. The composition of claim 1, wherein the oil agent is one or more of an oil ester, mineral oil, isopropyl myristate, isopropyl palmitate, isopropyl myristate dioctyl maleate, a fatty acid, or a salt or methyl, ethyl, propyl or butyl ester of a fatty acid.

11. The composition of claim 10, the oil agent has a concentration value in a range of 1 wt % to 15 wt % in the aqueous hair perm formulation.

12. The composition of claim 1, wherein a weight ratio of the emulsifier agent to the oil agent in the aqueous hair perm formulation is a value in a range from 1:1 to 4:1.

13. A method, comprising:
producing a permanent hair wave or texture, including:
providing a composition including an aqueous hair perm formulation, the aqueous hair perm formulation including an emulsion of:
an active agent capable of reducing disulfide cysteine bond linkages in hair,
an emulsifying agent, and
an oil agent; and
applying the aqueous hair perm formulation to a subject's hair.

14. The method of claim 13, further including, before the applying the aqueous hair perm formulation to the hair, placing the hair into a hair curling system, the hair curling system including structures for wrapping portions of the hair there-around and maintaining the portions of the hair in place; and then the applying of the aqueous hair perm formulation to the hair.

15. The method of claim 13 further including, after the applying of the aqueous hair perm formulation to the hair, adding an aqueous solution that includes a second active agent, the second active agent capable of increasing the disulfide cysteine bond linkages in the hair.

16. The method of claim 15, wherein the second active agent includes hydrogen peroxide.

17. The method of claim 15, wherein the applying of the aqueous hair perm formulation is for an application time value in a range from about 1 to 30 minutes.

18. The method of claim 15, wherein the applying of the aqueous hair perm formulation to a subject's hair is done while maintaining a temperature of the hair and the aqueous hair perm formulation at about an ambient temperature.

19. The method of claim 18, wherein the ambient temperature is in a range from about 60° F. to 98° F.
